(19) United States
(12) Patent Application Publication
(10) Pub. No.: US 2020/0222688 A1
(43) Pub. Date: Jul. 16, 2020

(54) ISOLATION OF A PASSIVATION

(71) Applicants: AESCULAP AG, Tuttlingen (DE); EBERHARD KARLS UNIVERSITÄT TÜBINGEN, Tübingen (DE)

(72) Inventors: BORIS HOFMANN, Balgheim (DE); MARKUS WESTERHAUSEN, Reutlingen (DE)

(21) Appl. No.: 16/638,956

(22) PCT Filed: Aug. 10, 2018
(86) PCT No.: PCT/EP2018/071772

§ 371 Date: Feb. 13, 2020

(30) Foreign Application Priority Data
Aug. 16, 2017 (DE) 10 2017 118 687.5

(51) Int. Cl. A61N 1/05; G01M 3/02; C23C 22/05; H01L 23/31; H01L 23/538; H01L 23/498; H01L 23/29

(52) U.S. Cl. CPC A61N 1/05; G01M 3/02; C23C 22/05; H01L 23/3192; A61N 1/36125; H01L 23/3121; H01L 23/4985; H01L 23/29; H01L 23/5387

(57) ABSTRACT

In a method for isolating a passivation, an isolating wall is arranged along the circumference of at least one opening created in a predetermined way in the passivation. A resultant passivation system has an electrically and/or fluid-tightly and/or gas-tightly isolating wall along the circumference of at least the opening created in a predetermined way in the passivation. The isolating wall of a predetermined thickness along a circumference of an opening produced in a predetermined way in a passivation of a medical implant advantageously provides an isolation of the passivation or components of the same with respect to electricity and/or liquid acting on the passivation or the components of the same from the opening.

### CROSS REFERENCE TO RELATED APPLICATIONS

This is the United States national phase entry of International Application No. PCT/EP2018/071772, filed Aug. 10, 2018, which claims the benefit of priority of German Application No. 10 2017 118 687.5, filed Aug. 16, 2017. The contents of International Application No. PCT/EP2018/071772 and German Application No. 10 2017 118 687.5 are incorporated by reference herein in their entireties.

### FIELD

The invention relates to an isolation of an artificially created opening in a passivation, and relates in particular to an electrically and/or fluid-tightly and/or gas-tightly isolating closure of an opening created in a predetermined way in a passivation without filling this opening completely, for instance, galvanically and/or closing it completely.

### BACKGROUND

For medical therapy and diagnosis methods, for instance, electrically active and/or flexible implants are known. Such implants are normally bio-stable, bio-compatible, and encapsulated in an electrically isolating way, and passivated, for instance, by means of a passivation layer or a multi-layer passivation system comprising inter alia a plurality of passivation layers and at least one intermediate layer arranged there between and contacted electrically.

In encapsulated, flexible implants openings in a passivation may enable an electrical access to the at least one electrically active intermediate layer in this passivation and/or between passivation layers or passivation tiers, and frequently the passivation over an electrode and/or an implant electrode is also opened by means of an opening which is created or produced artificially, i.e. in a predetermined way, so as to establish an electrical contact between the electrode and the tissue.

The artificially created opening, however, leaves the side wall of the opening vulnerable with respect to electrical influences and also negatively degrading influences. The defect in the passivation which is created artificially in the form of the opening can indeed be closed again, for instance, by the galvanic filling with an electrically conductive material, whereafter the electrode side wall is again protected from degradation and/or impairment (wetness, delamination, etc.) and also the electrode is still in contact with the tissue.

However, after the galvanic filling with electrically conductive material, conductivity also exists with respect to the side wall in the passivation, so that further electrically active layers are inevitably also connected electrically with the electrode (cf. FIG. 2, left half of the picture with electrically active faces of conductive material, for instance, metal faces, in a passivation which would, in the case of galvanic filling, for instance, be contacted electrically with respect to the electrode via a fluid). Such (necessarily) created openings in the passivation thus enable disadvantageously also an undesired electrical access to the at least one electrically active intermediate layer in the passivation.

### SUMMARY

An object underlying the invention is therefore to provide a method, a passivation system, and an isolating wall by means of which an opening in the passivation can at least be isolated electrically with respect to the passivation, and which enable the further use of electrically active components within the passivation.

The invention is based on the general idea of closing an artificially created opening in a passivating layer not (only) by means of galvanic filling, for instance, but to close this opening again by means of an at least electrically isolating side wall without having to close or refill it completely. In other words, in accordance with the invention, instead of the known galvanic filling and/or closing of the opening, the circumferential wall of the opening is provided with an electrically and/or fluid-tightly and/or gas-tightly isolating side wall or coating which prevents possible electrically active components in the passivation from being contacted with respect to an electrolyte (fluid) which charges the electrode of the implant via the opening which remains otherwise free. The side wall passivation proposed in accordance with the invention thus does not only constitute an (electrical and/or fluid-tight and/or gas-tight) isolation with respect to the passivation, but additionally protects same from further degrading effects, such as the direct subjecting and/or exposing of passivation layers and intermediate layers and/or corresponding parts of the multi-layer system to liquid. At the same time, the passivation is protected from the lateral penetration of fluids, such as for instance, water. The opening itself in the passivation is maintained and may be used for further applications, for instance, for a lead-through, a plug connection, or a header, or may be filled galvanically if required. Due to the coating of the side wall(s) of (electrode) openings with an electrically isolating layer the actual passivation layer may also be used electrically since it operates separately from an artificially created (electrode) opening, but is at the same time also protected from external influences which may act due to these openings.

In detail, a method for isolating a passivation comprises a step in which an isolating wall is arranged along the circumference of at least one opening created in a predetermined way in the passivation.

Preferably, the isolating wall is produced to be electrically isolating and/or fluid-tightly and/or gas-tightly isolating.

Preferably, the isolating wall extends from a lower end of the passivation through the passivation to an upper end of the passivation.

Preferably, in the afore-mentioned method the isolating wall is coated in at least one round-shaped, rectangular-shaped, elliptic or cone-shaped opening and preferably covering the entire side wall face of the opening in the passivation, wherein the passivation is a single-layer passivation or a multi-layer passivation system with at least one electrically active intermediate layer, and the isolating wall isolates in an electrical and/or fluid-tight and/or gas-tight manner said at least one electrically active intermediate layer and/or at least one electrode of a flexible or rigid medical implant which is bio-stable, bio-compatible and encapsulated in an electrically isolating manner as a bottom face of the opening.

Preferably, the isolating wall is produced in the method with a predetermined thickness and is configured to obtain, in the opening, an opening channel of a predetermined diameter in the passivation for a fluid supply to an electrode and/or for an accommodation of a lead-through, a plug connection and/or a header device, and to simultaneously keep electrically active components in the passivation electrically isolated with respect to the opening channel and/or protect same from penetration of the fluid in the passivation.

In accordance with a further aspect of the invention a passivation system comprises an isolating wall along the circumference of at least one opening created in a predetermined way in the passivation, said isolating wall being produced to be electrically and/or fluid-tightly and/or gas-tightly isolating.

Preferably, the isolating wall extends from a lower end of the passivation through the passivation to an upper end of the passivation.

Preferably, the isolating wall is arranged in at least one round-shaped, rectangular-shaped, elliptic or cone-shaped opening and covering the entire wall face of the opening in the passivation, wherein the passivation is a single-layer passivation or a multi-layer passivation system with at least one electrically active intermediate layer, and the isolating wall is arranged to isolate in an electrical and/or fluid-tight and/or gas-tight manner said at least one electrically active intermediate layer and/or at least one electrode of a flexible or rigid medical implant which is bio-stable, bio-compatible and encapsulated in an electrically isolating manner as a bottom face of the opening.

Preferably, the isolating wall is produced with a predetermined thickness and is configured to obtain, in the opening, an opening channel of a predetermined diameter in the passivation for a fluid supply to the electrode and/or for an accommodation of a lead-through, a plug connection and/or a header device, and to simultaneously keep electrically active components in the passivation electrically isolated with respect to the opening channel and/or protect same from penetration of the fluid in the passivation.

In accordance with a still further aspect of the invention an isolating wall of a predetermined thickness is provided along a circumference of an opening created in a predetermined way in a passivation of a medical implant, for isolating the passivation or components of the same with respect to electricity and/or liquid acting from the opening on the passivation or the components of the same.

### BRIEF DESCRIPTION OF THE DRAWING FIGURES

The invention will be described in detail in the following with further advantages and effects by means of preferred embodiments and with reference to the drawing figures, of which:

FIG. 1 shows a passivation, for instance, for a medical implant with an artificially created opening and an electrically isolating side wall arranged in the opening, in accordance with an embodiment;

FIG. 2 shows a three-dimensional scheme of the side wall passivation in accordance with the embodiment with electrically active faces in the passivation in a multi-layer system;

FIG. 3 shows a diagram for illustrating a demonstration of functioning of an isolating side wall in accordance with the embodiment;

FIG. 4 shows an example of a plug connection (header) with a side wall passivation; and

FIG. 5 shows an example of a substrate with opened electrodes and layers of at least electrically conductive material in the passivation in accordance with the embodiment.

The same or functionally equivalent features are provided with the same reference numbers in the individual Figures and are expediently not described redundantly. The Figures need not necessarily be considered as being to scale. A restriction to measuring units indicated in the Figures does not exist.

### DETAILED DESCRIPTION

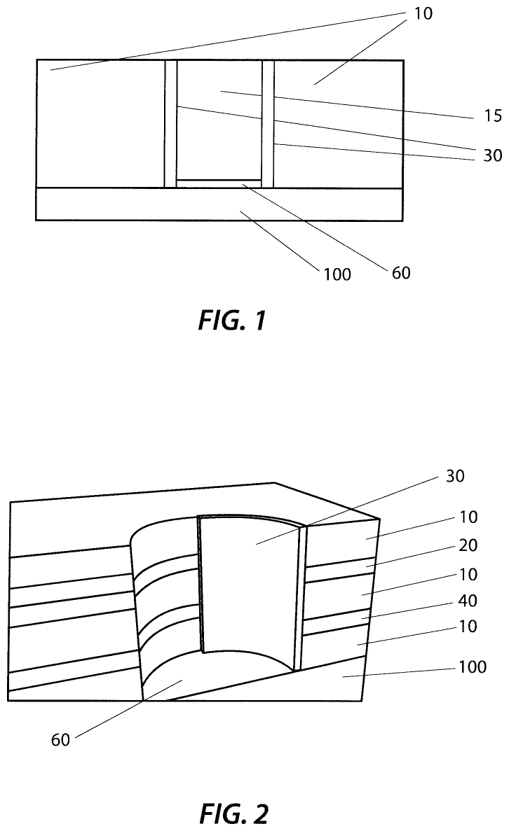

FIG. 1 shows in excerpts and schematically simplified a passivation, for instance, for a medical implant with an artificially created opening and an electrically isolating side wall arranged in the opening, in accordance with an embodiment.

As is shown in FIG. 1, an electrode and/or implant electrode 60 and a passivation 10 surrounding same are arranged on a substrate 100 which may form part of the implant that is not illustrated further. The electrode 60 is moreover arranged in the opening 15 created artificially, i.e. in a predetermined way, in the passivation 10 and forms a bottom face there. At the circumference and/or the (inner) wall of the opening 15, within the opening 15 and starting out from the electrode 60 and/or a lower side and/or a lower end of the passivation 10, an isolating layer 30 is produced with a predetermined thickness which extends to an upper side and/or an upper end of the passivation 10.

In this embodiment the shape and the size of the opening 15 are not restricted to a particular number, shape and size, but these parameters may be determined for the specific use. For instance, one opening 15 may be arranged, or a plurality of openings 15 of equal or different sizes may be arranged at equal or different distances to each other. The opening itself may, for instance, be formed in a round shape and/or cylindrically, a rectangular shape including a square shape, elliptically, or conically tapering toward the top or the bottom. It is to be understood that further suitable shapes of opening may also be conceivable for the specific use.

Neither are there any particular restrictions in accordance with this embodiment with respect to the procedure of application of the isolating layer 30 as long as it is a matter of an application or coating method which is admitted and/or compatible and altogether suitable for an intended use, for instance, from the field of medical implants.

The isolating layer 30 itself is advantageously and preferably at least electrically isolating and/or fluid-tightly isolating and/or gas-tightly isolating. In a modification, properties of this kind may be produced or varied (increased or decreased) site-specifically, i.e. for instance, by means of a multiple application of corresponding layers and/or a thickness variation of the isolating layer 30 along the length of extension thereof. Altogether, the isolating layer 30 forms an at least electrically isolating side wall and/or electrical side wall isolation in the artificially created opening 15 in the passivation 10 and results in a passivation with an electrically passivated side wall.

FIG. 2 shows in excerpts and schematically a three-dimensional scheme of the side wall passivation pursuant to the embodiment with an isolating barrier with respect to electrically active faces within the passivating layer and/or in the passivation in a multi-layer system.

In detail, FIG. 2 shows a section from a multi-layer passivation system on the substrate 100 with the (implant) electrode 60 which is protected by the passivation system from various negative degradation effects. The multi-layer passivation system forms, with its passivation layers and/or passivation tiers 10, here an upper or first, a middle or second, and a lower or third passivation layer, and sandwiched electrically active intermediate layers 20, 40, here an upper or first intermediate layer 20 (Autop) and a lower or second intermediate layer 40 (Aubottom) of an electrically conductive material, for instance, a metal or a semiconductor, a passivating multi-layer system. FIG. 2 also shows the opening 15 (the reference number has been omitted in the Figure for convenience) and a partial section of the isolating wall 30 at the circumference of the opening 15.

The side wall passivation shown in FIG. 2 does not only constitute an (electrical) isolation with respect to the passivation, but protects the same additionally from further degrading effects, such as for instance, a direct charging by liquid present in the opening 15. As results from FIG. 2, left part, an unprotected side wall in the passivation would, in the case of a conventional (galvanic) filling of the artificially created defect in the passivation in the form of the opening 15, inevitably also be connected electrically with the electrode 60.

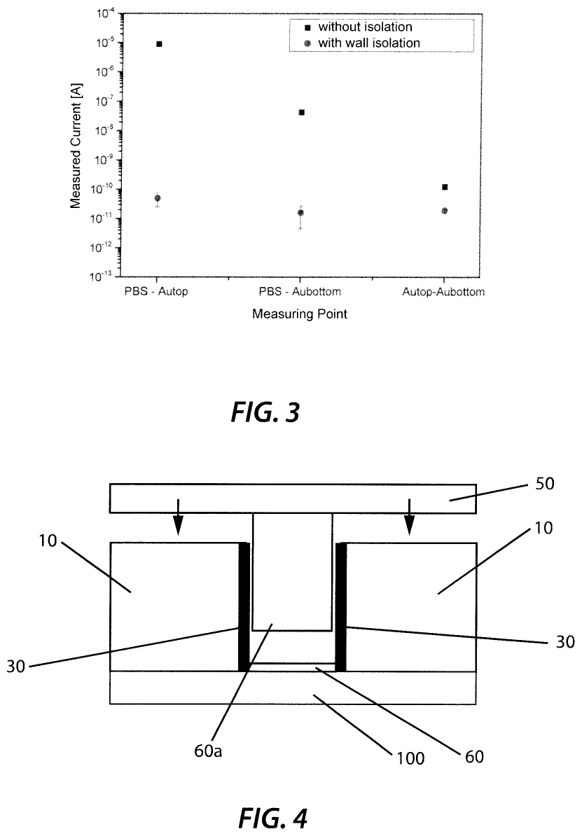

FIG. 3 shows a diagram for illustrating a demonstration of functioning of the isolating side wall pursuant to the embodiment. For a functional demonstration, in this embodiment a current (direct current, 1V applied; thus directly reciprocal to the resistance) from a (non-illustrated) reference electrode which was stored in a (non-illustrated) PBS bath over the unprotected electrode opening 15, i.e. not having an isolating wall, was measured with respect to the two intermediate layers 20, 40 (Autop, Aubottom; cf. FIG. 2) of conductive material. Then, the side wall of the electrode opening 15 was electrically isolated by means of a physical atom layer deposition with a 100 nm thick Ti02 layer and the afore-described measurement was performed again. PBS stands, for instance, for Phosphate Buffer Solution or Phosphate Buffered Saline. PBS and its applicability are known and are therefore not described redundantly here.

All in all it may be seen in FIG. 3 that the electrical resistance, after applying or coating the isolation on the wall of the electrode opening 15, increases by magnitudes and correspondingly the current measured while the isolating wall is present (lower row of dots in FIG. 3) decreases with respect to the non-isolated wall to values of approx. 10⁻¹⁰ to 10⁻¹¹ A. With such values the side wall has to be considered to be electrically isolated.

FIG. 4 shows in excerpts and schematically an example of a plug connection (header) with a side wall passivation. Since, pursuant to the present embodiment, an opening 15 remains in the passivation 10 which is reduced merely by the thickness of the isolating wall and/or coating, this opening 15 may be utilized for further uses, for instance, for a lead-through, a plug connection or a header 50 with a section 60a insertable in the opening 15 and contacting the electrode 60, indicated by arrows in FIG. 4.

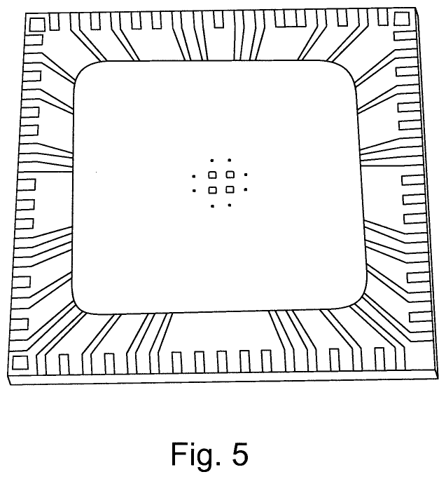

FIG. 5 illustrates an example of a practically usable substrate with opened electrodes and layers of electrically conductive material in the passivation pursuant to the embodiment. The electrically isolating side wall passivation enables particularly advantageously the manufacturing of (flexible) implants with layers or faces of electrically conductive material, for instance, metal faces, in the passivation, as illustrated in FIG. 5.

Several openings 15 of various sizes, arranged in a cross shape with larger openings 15 inside and comparatively smaller openings 15 outside, may be recognized in FIG. 5. The opened electrodes 60 in the middle of the substrate are isolated electrically from the faces of electrically conductive material and can thus be used individually.

As was described before, in a method for isolating a passivation, an isolating wall is arranged along the circumference of at least one opening created in a predetermined way in the passivation. A resultant passivation system comprises an electrically and/or fluid-tightly and/or gas-tightly isolating wall along the circumference of at least the opening created in a predetermined way in the passivation. The isolating wall of a predetermined thickness along a circumference of an opening created in a predetermined way in a passivation of a medical implant provides advantageously an isolation of the passivation or components of the same with respect to electricity and/or liquid acting on the passivation or the components of the same from the opening.

By coating the side walls of (electrode) openings with an electrically isolating layer, manifold examples of use result. For instance, the actual passivation layer may also be used electrically since it works separately from an artificially created (electrode) opening, but is at the same time also protected from external influences which may act due to these openings.

It is to be understood that the invention is not restricted to a concrete isolating layer in an electrode opening of an encapsulation and/or passivation structure at an implant, but that various configurations, equivalent embodiments and modifications are conceivable.

Moreover, it is to be understood that the described embodiments and the drawings which are not to scale only have an exemplary character and that modifications may readily result for the person skilled in the art without the scope of the description being left. Likewise, external shapes, dimensions and the like are not subject to any particular restrictions as long as the effect and the functionality in accordance with the invention are provided and achieved by them.

1. A method for isolating a passivation, wherein an isolating wall is arranged along the circumference of at least one opening created in a predetermined way in the passivation.

2. The method according to claim 1, wherein the isolating wall is produced to be electrically isolating and/or fluid-tightly and/or gas-tightly isolating.

3. The method according to claim 1, wherein the isolating wall extends from a lower end of the passivation through the passivation to an upper end of the passivation.

4. The method according to claim 1, wherein the isolating wall is coated in at least one round-shaped, rectangular-shaped, elliptic or cone-shaped opening in the passivation, wherein the passivation is a single-layer passivation or a multi-layer passivation system with at least one electrically active intermediate layer, and the isolating wall isolates in an electrical and/or fluid-tight and/or gas-tight manner said at least one electrically active intermediate layer and/or at least one electrode of a flexible or rigid medical implant which is bio-stable, bio-compatible and encapsulated in an electrically isolating manner as a bottom face of the opening.

5. The method according to claim 1, wherein the isolating wall is produced with a predetermined thickness and is configured to obtain, in the opening, an opening channel of a predetermined diameter in the passivation for a fluid supply to an electrode and/or for an accommodation of a lead-through, a plug connection and/or a header device, and to simultaneously keep electrically active components in the passivation electrically isolated with respect to the opening channel and/or protect electrically active components in the passivation from penetration of the fluid in the passivation.

6. A passivation system comprising an isolating wall produced to be electrically isolating and/or fluid-tightly and/or gas-tightly isolating along the circumference of at least one opening created in a predetermined way in the passivation.

7. The passivation system according to claim 6, wherein the isolating wall extends from a lower end of the passivation through the passivation to an upper end of the passivation.

8. The passivation system according to claim 7, wherein the isolating wall is arranged in at least one round-shaped, rectangular-shaped, elliptic or cone-shaped opening and covering the entire wall face of the opening in the passivation, wherein the passivation is a single-layer passivation or a multi-layer passivation system with at least one electrically active intermediate layer, and the isolating wall is arranged to isolate in an electrical and/or fluid-tight and/or gas-tight manner said at least one electrically active intermediate layer, and/or at least one electrode of a flexible or rigid medical implant which is bio-stable, bio-compatible and encapsulated in an electrically isolating manner as a bottom face of the opening.

9. The passivation system according to claim 6, wherein the isolating wall is produced with a predetermined thickness and is configured to obtain, in the opening, an opening channel of a predetermined diameter in the passivation for a fluid supply to the electrode and/or an accommodation of a lead-through, a plug connection and/or a header device, and to simultaneously keep electrically active components in the passivation electrically isolated with respect to the opening channel and/or protect electrically active components in the passivation from penetration of the fluid in the passivation.

10. An isolating wall of a predetermined thickness along a circumference of an opening created in a predetermined way in a passivation of a medical implant, for isolating the passivation or components of the same with respect to electricity and/or liquid acting on the passivation or the components of the same from the opening.
